(19) United States
(12) Patent Application Publication
(10) Pub. No.: US 2019/0029880 A1
(43) Pub. Date: Jan. 31, 2019

(54) Devices and Methods for Treating Epistaxis

(71) Applicant: Brian R. DuBois, Redwood CIty, CA (US)

(72) Inventor: Brian R. DuBois, Redwood CIty, CA (US)

(21) Appl. No.: 16/045,923

(22) Filed: Jul. 26, 2018

Related U.S. Application Data
(60) Provisional application No. 62/537,404, filed on Jul. 26, 2017.

(51) Int. Cl. A61F 7/12

(52) U.S. Cl. CPC A61F 7/12; A61F 13/2005

(57) ABSTRACT

An exemplary medical apparatus includes a heated nasal insert, where the nasal insert is configured to heat tissue to be treated to a temperature in the range of substantially 46° C. to substantially 52° C. in use. An exemplary medical method for inducing hemostasis in tissue of a patient includes possessing a nasal insert; apposing the nasal insert to tissue of a patent; heating said nasal insert to a temperature that causes tissue of a patient to be heated to a temperature in the range of substantially 46° C. to substantially 52° C.; and maintaining the tissue of a patent treaded by said nasal insert within a temperature range of substantially 46° C. to substantially 52° C.

This application claims priority to U.S. Provisional Patent Application Ser. No. 62/537,404, filed on Jul. 26, 2017, which is herein incorporated by reference in its entirety.

### FIELD OF THE INVENTION

The invention generally relates to devices and methods for the treatment of epistaxis, and more particularly to devices and methods for treating epistaxis using a power supply to heat and maintain a nasal insert at a therapeutic temperature.

### BACKGROUND

Tamponade treatment for epistaxis is painful and traumatic to the nasal mucosa, and may necessitate hospitalization for several days. Epistaxis is the clinical term for what laypeople refer to as nosebleed. A posterior pack is placed to occlude the choanal arch and, in conjunction with an anterior nasal pack, provide hemostasis. Posterior packing can be accomplished with gauze, a Foley catheter, a nasal sponge/tampon, or an inflatable nasal balloon catheter. Posterior packing is very uncomfortable and may necessitate procedural sedation.

Hot-water irrigation (HWI) was introduced as a treatment of epistaxis more than 100 years ago. The treatment for posterior epistaxis involved running hot water through the bleeding nose cavity, and the treatment was successful in many cases. The therapeutic temperature of the hot water is from 46° C. to 52° C. Water temperatures below 46° C. have no effect, only light changes occur at 46° C. and 47° C., and the best effect occurs between 48° C. and 52° C. Vasodilation, edema of the mucosa, and subsequent narrowing of the intranasal lumen occur at a temperature of 48° C. or higher. Severe changes including epithelial necrosis, occur when the treatment temperature is 53° C. or higher. The results of the study indicate that the hemostatic effect of hot water treatment for epistaxis may be caused by: (1) vasodilation of the mucosal vessels which slows bleeding rate, (2) edema and narrowing of the intranasal lumen, and (3) cleaning of the nose from blood coagulates; it is theorized that the elevated temperature accelerates the clotting cascade. In a study conducted by Stangerup et. al. in 1999, the treatment proved to be effective, less painful, and less traumatic, and required a shorter hospital stay than tamponade treatment. In the study performed by Stangerup et. al., a thermometer (0° C.-100° C.), a thermo-bucket filled with fresh water (50° C.) from the hot water tap, a 10-mL and a 100-mL syringe, and the catheter. The patient was instructed to sit with the head flexed and a catheter was introduced via the bleeding nasal cavity. The balloon was then filled with 10 mL of hot water, and the catheter was pulled back so that the balloon on the end of the catheter sealed the posterior choana of the bleeding nasal cavity. The nasal cavity was irrigated forcefully via the catheter with 500 mL of hot water using a 100-mL syringe. After irrigation, the catheter was removed and the patient was observed for 15 minutes.

Another method for causing hemostasis is to apply warm water to the bleeding site in the range of 46° C. to 52° C. It is not fully understood why warm water causes hemostasis, however, it has been theorized that the water washes away blood clots, the warm temperature initially dilates the blood vessels which slows bleeding, then tissue edema around the blood vessel(s) results in constriction of the bleeding vessel. Finally, it is theorized that the elevated temperatures may accelerate the hemostatic cascade.

A problem with applying warm water to cause epistaxis is the uncontrolled flow of bloody water from the operative site, creating a biohazardous mess and potentially frightening the patient. The water may cause discomfort or safety issues by being aspirated into the lungs, it may flow into sinuses, or it may exit the patient in an uncontrolled manner or cause bloodborne safety issues to the caregiver. Another problem is that warm water in the therapeutically effective range is initially too hot for many patients, and causes pain and discomfort unless the irrigation is started at a lower temperature, such as 42° C., and then increased to a therapeutically effective temperature. However, increasing water temperature over time extends the treatment duration, and does not address the other problems associated with HWI treatment.

Although the Hot Water Irrigation treatment used by Stangerup et. al. was effective, it is not commonly used because it is inconvenient and time consuming for the care giver. Therefore, a better method and devices are described herein to provide convenience, reduce time, and improve patient comfort.

Balloon pressurized devices also have been used to arrest bleeding inside body cavities. These devices consist of a balloon mounted on a tubular catheter. The balloon is inserted into a bleeding body cavity, (such as a nasal cavity), and the balloon is inflated. The balloon presses against the source of bleeding and assists the clotting of the blood in order to create hemostasis.

A considerable improvement to the devices is made when the balloon is covered by a hemostatic agent such as the device disclosed in international patent application PCT/GB98/01732, the disclosure of which is incorporated herein by reference in its entirety. This patent application discloses a device suitable for treating a bleeding diverticulum or other body cavity. One example of such a hemostatic agent is a tubular knitted fabric manufactured from carboxymethylated cellulose which is reinforced with nylon. The carboxymethylated cellulose swells and turns into a gel when in contact with blood or mucus fluid; the resultant gel acts as a strong hemostatic agent. The nylon reinforcement maintains the integrity of the fabric after the gelling takes place. This principle is used in the Rapid Rhino.™ device currently marketed by Smith and Nephew and is used to treat nose bleeding. A similar device based on the same principle is designed to treat bleeding in diverticula in the colon.

There is a technical problem with the aforementioned inflatable balloon devices because, in use, it is difficult to control accurately the pressure within the balloon. On the one hand, the pressure must be high enough to control the bleeding in the body cavity, whereas on the other hand if the pressure is too high then this may result in serious damage to the body cavity; i.e., it may induce trauma. For example, when treating a bleeding nose a physician must exercise extreme care so as not to damage the sensitive mucus tissue in contact with the device. Such damage may result in toxic shock syndrome. The risk of trauma may even deter certain physicians from using these devices even though their efficacy is well proven. In known devices, the physician typically has to control the pressure in the balloon in one of two ways:
1. By inflating the balloon with a measured set volume of an inflation medium, such as air, using for example a calibrated syringe. However, this set volume method does not take into account the differing sizes of body cavities. Consequently, in the same surgical procedure a smaller than average cavity may be subjected to excessive pressure which could result in undesirable complications. Moreover, this method relies completely on the skill and diligence of the physician.
2. A second method involves inserting a safety cuff or pilot balloon, such as a second inflatable balloon, in the inflation line between the active balloon and the syringe. The safety cuff remains outside the body cavity and the physician monitors the pressure in the active balloon by manually feeling the safety cuff during inflation of the active balloon. Hence, this method relies on the experience of the physician, particularly his or her tactile ability in feeling the correct level of pressure.

The above problems are exacerbated when inflatable balloon devices are used which are made from elastomeric materials such as silicon rubber. With an elastomeric balloon a high initial pressure is required to expand the balloon. As the balloon expands the thickness of the balloon wall decreases. This results in a decrease in force within the wall of the balloon. The effect of the balloon elasticity therefore results in a nonlinear relationship between pressure and balloon volume, which increases the problems facing the physician.

Accordingly, there is need for improved methods and apparatus for treating a bleeding body cavity, in particular a bleeding nose or diverticula.

### SUMMARY

An exemplary medical apparatus includes a nasal insert, which includes a flexible core; at least one resistive wire associated with the flexible core; and a cover over at least part of the flexible core and the at least one resistive wire; where the nasal insert is configured to radiate heat in the range of substantially 46° C. to substantially 52° C. in use.

An exemplary method for inducing hemostasis in tissue of a patient includes possessing an insert, which includes a flexible core, at least one resistive wire associated with the flexible core, and a cover over at least part of the flexible core and the at least one resistive wire; apposing the insert to tissue of a patent; causing electrical power to flow through the resistive wire; and controlling at least one characteristic of the electrical power to maintain the temperature of the insert in the range of substantially 46° C. to substantially 52° C.

The characteristics and utilities of the present invention described in this summary and the detailed description below are not all inclusive. Many additional features and advantages will be apparent to one of ordinary skill in the art given the following description. There has thus been outlined, rather broadly, the more important features of the invention in order that the detailed description thereof that follows may be better understood, and in order that the present contribution to the art may be better appreciated.

### BRIEF DESCRIPTION OF THE DRAWINGS

FIG. 1 is a perspective view of a nasal insert system, including a nasal insert shown with its cover cut away, and a control unit.

FIG. 2 is a cutaway perspective view of the control unit of FIG. 1.

FIG. 3 is a perspective view of the nasal insert system of FIG. 1 in use, with the head of a patient shown as a cutaway view to allow the nasal cavity to be seen.

The use of the same reference symbols in different figures indicates similar or identical items.

### DETAILED DESCRIPTION

The present invention relates to apparatus and methods for arresting bleeding in a body cavity such as intra-nasal bleeding (epistaxis). The present invention also relates to the control of bleeding in many other body cavities, chambers or conduits; for example, bleeding diverticula in the colon, upper gastrointestinal bleeding in the stomach or duodenum, bleeding in the esophagus, and bleeding in the uterus; the device and method not limited to any particular body cavity or treatment location. The size, dimensions, and characteristics of the invention herein may be altered according to the treatment site or body cavity.

Various devices are used for treating bleeding inside the body. Examples for the control of epistaxis include a tampon material which expands when wetted and balloon devices which apply pressure to the source of the bleeding.

Tampons may consist of a polymeric material which is compressed to a relatively small size when dry, and which expand when wetted. In the process of expanding they absorb blood and fluid and apply light pressure to the source of the bleeding.

The present invention provides methods and devices for increasing the temperature of body tissues to the proper temperature range that will cause hemostasis.

The present invention provides methods, devices, and apparatus for arresting bleeding in a body cavity comprised of a nasal insert with a hemostatic fabric soaked in fluid such as warm water and a resistive heating element, or its equivalent, and one or more thermocouples that is connectable to a portable electronic device that provides power to maintain the temperature of the nasal insert in the range of 46° C. to 52° C.

Temperatures in the range of 46° C. to 52° C. are ideal for inducing hemostasis without causing tissue necrosis. Temperatures below 46° C. may not produce hemostasis whereas temperatures of 53° C. and higher are known to cause tissue necrosis.

According to a first embodiment, a nasal insert may be comprised of an outer material that will absorb or hold water such as a fabric, or an equivalent as will be recognized by those skilled in the art. The outer material may be used as a covering over a heating element. Electrical energy is used to raise the temperature of the heating element and the outer material that is soaked in fluid sufficient to bring the temperature of the outer material and fluid into the therapeutic range of 46° C. to 52° C. The fluid held in the fabric may be absorbed by the tissue resulting in edema during the therapy.

A second embodiment may further include one or more thermocouples or other temperature sensing devices that are positioned integral to the nasal insert and provide feedback to the power source that is supplying the electrical energy to ensure that the nasal insert temperature stays in the correct therapeutic range. Other means of heating, detecting, and controlling the temperature of the fluid absorbed into the material and in contact or near contact with the tissue surface will be recognized by those skilled in the art and are part of the invention herein across the various embodiments.

The nasal insert may be soaked in fluid before, during, or after insertion, and positioned at the bleeding site, maintained at the therapeutic temperature and left for an adequate period of time to cause cessation of bleeding. After the bleeding stops, the nasal insert may be removed from the operative site.

A similar embodiment may further incorporate a balloon containing the heating element that is inflated with a fluid and the entire device maintained at the therapeutic temperature.

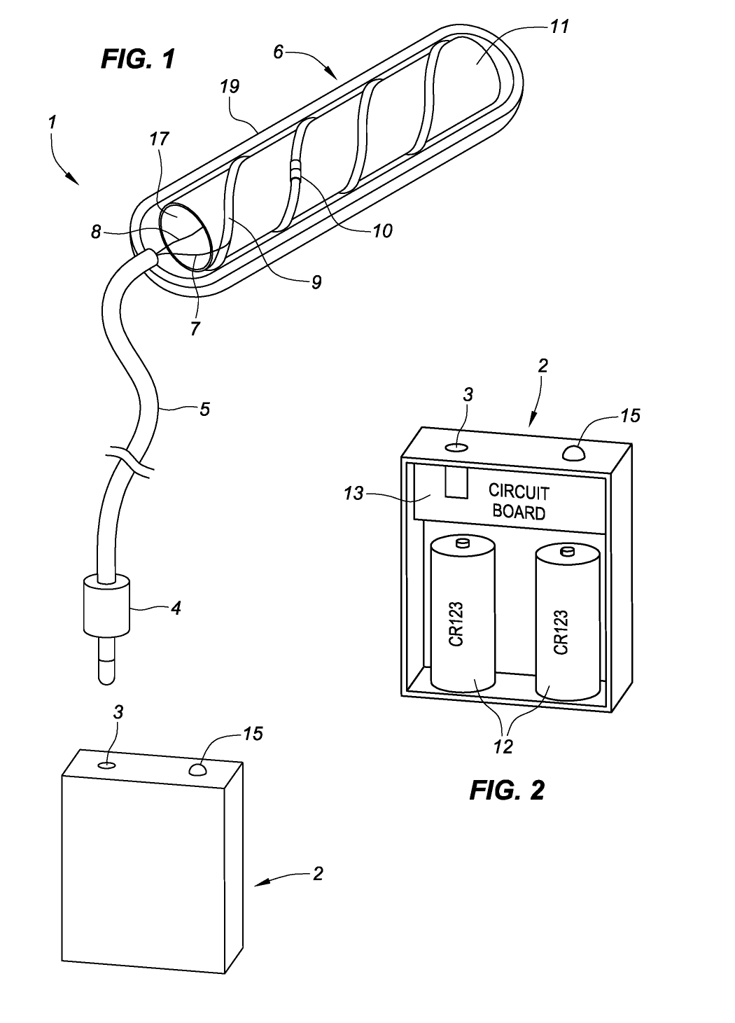

Referring to FIG. 1, a nasal insert system 1 includes a nasal insert 6 that is connected to a control unit 2. Advantageously, the nasal insert 6 is detachably connected to the control unit 2 such that the control unit 2 may be reusable, and the nasal insert 6 itself may be a disposable device. However, the nasal insert 6 may be fixed to the control unit 2, such that the entire nasal insert system 1 is a disposable one-time use device.

The nasal insert 6 includes a resistive wire 9 wrapped around a core 11. The resistive wire 9 may be fabricated from any suitable material that produces head when electricity passes through it, such as nickel-chromium alloy. The core 11 may be fabricated from a soft, flexible, malleable or foam material, or from any other suitable material that is capable of insertion into the nasal cavity of a patient without damaging tissue or causing pain. For example, the core 11 may be fabricated from a flexible material such as but not limited to polyethylene, polytetrafluoroethylene(PTFE), or polyvinyl chloride (PVC). The core 11 provides structure to the nasal insert 6 and provides a structure around which the resistive wire 9 is wrapped. Alternately, the resistive wire 9 may be embedded in the core 11, rather than wrapped around that core 11. The core 11 may be a tube that includes a lumen 17 defined at least partially along its length in order to receive one or more wires 8 carrying power from the control unit 2. The wires 8 may be connected to opposite ends of the resistive wire 9 in order to complete the circuit.

The nasal insert 6 optionally may be configured to sense temperature in any suitable manner. As one example, at least one thermocouple 10 may be wired in series with the resistive wire. The thermocouple 10 may include two or more wires fabricated from dissimilar metals that are electrically connected to one another either by contact or by welding them together. However, any suitable thermocouple 10 may be used. Alternately, a temperature sensor other than a thermocouple 10 may be used to measure the temperature of the nasal insert 6 in use, such as but not limited to a thermistor or a resistive temperature detector (RTD).

A cover 19 may cover the core 11, resistive wire 9, and/or other components of the nasal insert 6. The cover 19 may act to diffuse the heat from the resistive wire 9 to reduce or eliminate hot spots, and may facilitate entry of the nasal insert 6 into the nasal cavity. As one example, the cover 19 may be a fabric or other material that is hydrophilic, such that the cover 19 may be wetted with water, saline solution or other liquid prior to insertion in order to reduce friction during insertion. The cover 19 may be fabricated from a material such as carboxymethyl cellulose, polyethylene vinyl alcohol, calcium alginate, chitosan, or cotton that absorbs water when the cover 19 is soaked prior to insertion into the nose. The water lubricates the cover 19 to provide a slippery surface to improve patient comfort during insertion of the nasal insert 6. The water absorbed by the cover 19 may provide water for osmosis into apposed tissues to enable edema of the tissues. As another example, the cover 19 may be fabricated from material with hemostatic properties, as described in greater detail below. In this way, the cover 19 itself may facilitate treatment of epistaxis, in conjunction with the heat treatment applied by the nasal insert 6. The cover 19 may be secured to the core 11 with adhesive such as cyanoacrylate. Alternately, the cover 19 may be affixed to, secured to, or held relative to the core 11 in any other suitable manner, such as by stretching the cover 19 over the core 11 and then shrink-fitting the cover 19 to the core 11 or by cinching an opening after the core 11 is inserted into the cover 19 through that opening.

A cord 5 may extend from the nasal insert 6, terminating in a plug 4 that is received by a corresponding plug receptacle 3 in the control unit 2. The cord 5 is electrically conductive and acts to transmit electric power to the nasal insert 6. The plug receptacle 3 may be a standard 2.5 mm coaxial plug, a USB plug, or any other suitable electrical plug receptacle, and the plug 4 corresponds to the particular configuration of the plug receptacle 3. Where the 2.5 mm coaxial plug receptacle 3 is used, the cord 5 includes two wires (not shown) extending between the nasal insert 6 to provide a closed electrical circuit between the control unit 2 and the nasal insert 6. Where the USB plug receptacle 3 is used, the cord 5 includes two wires 8 extending between the nasal insert 6 to provide a closed electrical circuit between the control unit 2 and the nasal insert 6, and optionally may include one or more sensor wires 7 that connect the thermocouple 10 or other temperature sensor to the control unit 2 for transmitting temperature data to the nasal insert 6.

The control unit 2 may include a status indicator 15 to indicate whether the nasal insert system 1 is on and/or operating. The status indicator 15 may be a light emitting diode (LED), and may illuminate in one or more colors to provide information regarding the status of the therapy and controller. For example, the LED may emit green light when the device is on and operating correctly; the LED may then change to yellow when the therapy is completed, and the LED may illuminate red when there is a problem or fault. The status indicator 15 may also flash on and off to provide information to the user or patient about the status of the nasal insert system 1 and/or the therapy. Alternately, the status indicator 15 may include two or more different LEDs, a data display screen, or one or more other indicators of the status of the nasal insert system 1. Alternately, the status indicator 15 may be omitted. Alternately, the status indicator 15 may be provided on the plug 4 or on another location associated with the nasal insert 6.

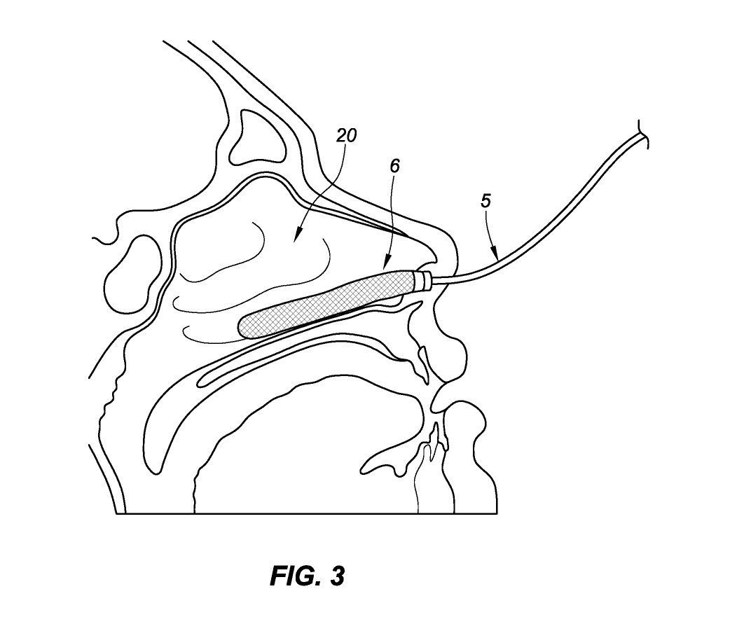

Referring also to FIG. 2, the control unit 2 includes at least one battery 12. As one example, the control unit 2 includes two replaceable 3V CR123 lithium batteries. As another example, the control unit 2 includes at least one built-in rechargeable battery that can be recharged by the clinician after each use. As another example, the control unit 2 may be connected to power from a wall socket via an AC adapter, in addition to or instead of the use of a battery 12, in order to recharge the battery and/or to provide therapy while drawing power from an external power source. The at least one battery 12 is connected to the wires 8 in a conventional manner.

The control unit 2 also includes at least one controller 13 that is electrically connected to and powered by the at least one battery 12 in a conventional manner. The controller 13 may include an integrated circuit (IC), field-programmable gate array (FPGA), printed circuit board (PCB), memory storage such as RAM, and/or any other suitable components. The controller 13 controls the temperature of the nasal insert 6 and provides timing logic functions for the nasal insert system 1. In some embodiments, the controller 13 is configured to power on and cause the flow of electrical power to the nasal insert 6 when the plug 4 is inserted into the plug receptacle 3 to complete an electrical circuit. In other embodiments, the controller 13 is configured to power on and commence operation upon actuation of a switch (not shown) that connects power from the at least one battery 12 to the controller 13.

The controller 13 stores instructions for the operation of the nasal insert system 1. Such instructions are specific to the operation of this particular special-purpose nasal insert system 1. As one example, the controller 13 includes a timing function that initiates therapy by switching on transmission of power through the wires 8 to the resistive wire 9, and then stops the therapy after a predetermined time by switching off transmission of power through the wires 8 to the resistive wire 9. As another example, the controller 13 may control the rate of power flow through the wires 8 to the resistive wire 9 to increase the temperature of the nasal insert 6 gradually at a rate that is comfortable for the patient. Optionally, that rate may be controllable. As another example, the controller 13 periodically stops the flow of electricity through the thermocouple 10 and measures the voltage across the thermocouple 10 to measure temperature of the nasal insert, then restarts the flow of electricity to control the temperature of the nasal insert 6 to maintain the therapeutic temperature range. Due to thermal inertia, the brief period of time that electricity does not flow to the resistive wire 9 during temperature measurement does not results in appreciable temperature loss at the nasal insert 6. As another example, the controller 13 may adjust the voltage or amperage of power transmitted to the resistive wire 9 based on those periodic temperature measurements, in order to maintain the nasal insert 6 at a temperature within the ideal therapeutic range. Optionally, the controller 13 includes wireless connectivity to allow transmission of data to and from the controller 13. In other embodiments, the control unit 2 is configured to include two plug receptacles 3 and control two separate nasal inserts 6 for simultaneous bilateral therapy of a patient using two separate nasal inserts 6 each connected to a separate plug receptacle 3. In such embodiments, the controller 13 is configured to control independently each separate nasal insert 6. Optionally, the electronica power supply 4 may receive commands wirelessly, through a touchscreen, through mechanical buttons, or in any other manner to allow the clinician to manually adjust one or more therapy options, such as but not limited to (i) therapy time, (ii) therapy set temperature range, and (iii) unilateral or bilateral therapy.

According to other embodiments, the cover 19 may include a hemostatic agent that retards or prevents bleeding and that is used in concert with the heated nasal insert. The term “hemostatic agent” means a substance that is capable of arresting, stemming or preventing bleeding by means other than inducing tissue growth alone. In other words, it is not tissue growth alone which is responsible for retarding or preventing bleeding. It will of course be appreciated that the hemostatic agent may have the beneficial property of inducing tissue growth in addition to its retardation or prevention of bleeding property. Preferably, the hemostatic agent is a bioactive compound or composition which causes vasoconstriction and/or blood coagulation. The list of potential hemostatic agents consistent with the invention herein will be recognized by those skilled in the art. A non-exhaustive list of examples of preferred hemostatic agents that retard or prevent bleeding include oxidized cellulose, such as Tabotamp™ oxidized cellulose, sold by Johnson and Johnson of One Johnson & Johnson Plaza, New Brunswick, N.J. 08933; calcium alginate, gelatin, chitosan, or collagen. A particularly preferred agent is carboxymethylated cellulose which can be purchased from Courtaulds Special Fibres, PO Box 111, 101 Lockhurst Lane, Coventry, England, CV6 5RS. Combinations of different agents may be used within the scope of the invention.

Preferably, the hemostatic agent that retards or prevents bleeding is provided in the cover 19, which in these embodiments may be in the form of a net or knitted, especially a weft knitted, textile material that envelopes the core 11. Alternately, the net or knitted textile material is fixed to the core 11. Alternatively, the hemostatic agent that retards or prevents bleeding is provided in the form of a flexible film that coats the outer surface of the balloon. The flexible film or other hemostatic agent may be adjacent or contacting the surface of the core 11 or positioned outside of subsequent layers of various materials. In such embodiments, the control unit 2, wires 8, resistive wire 9, and plug 4 may be omitted, such that the nasal insert 6 treats the nasal cavity without the use of electrical heating. Alternately, the hemostatic agent is used in conjunction with the treatment of the nasal cavity with the nasal insert system 1 such that the hemostatic agent associated with the cover 19 complements the electrical heating of the nasal cavity.

According to other embodiments, the temperature of the nasal insert 6 may be raised to a temperature within the therapeutically effective range by using an exothermic chemical reaction. One or more chemicals are placed inside the cover 19, and the nasal insert 6 is inserted into the patient's nasal cavity. A chemical reaction is initiated prior to or following insertion that produces heat and maintains the temperature of the nasal insert 6 within the therapeutic range of 46° C. to 52° C. for a period of time required to produce the therapeutic effect. Alternately, the nasal insert 6 is composed, in whole or in part, of the chemical or chemicals used to generate an exothermic reaction. Alternately, one or more chemicals are used to generate an exothermic reaction in addition to the use of electricity to heat the nasal insert system 1, such that electrical power is applied to the resistive wire 9 in order to fine-tune the temperature of the nasal insert 6 and/or to supplement the heat generated by the exothermic reaction.

An example of an exothermic chemical reaction that may be used to elevate the cover 19 or other outer material of the nasal insert 6 to the therapeutic temperature range is oxidation of iron. Oxygen in the air reacts with iron powder to yield iron oxide (rust) and heat. Iron powder is produced by crushing iron or spraying a molten stream with water. A mixture of iron powder, vermiculite, activated charcoal, water, and salt may be combined in a microporous pouch. In this embodiment, the nasal insert 6 includes a lumen defined therethrough, through which air is capable of flowing. Additionally, the patient may be able to breathe through the lumen. The internal lumen is lined with a microporous material configured to allow air and/or moisture to communicate with the chemicals in the insert thereby causing the exothermic reaction to occur. The cover 6 may be configured substantially as described above. The iron powder and other chemicals are located within the nasal insert 6, between the lumen and the cover. The activated charcoal is a porous material that has a surface area to as much as 2,000 square meters per gram. The activated charcoal may hold the water necessary for the oxidizing reaction to occur, and it is also thermally conductive to spread heat generally evenly. A variety of salts may be used, such as but not limited to sodium chloride. The salt acts as a catalyst to the oxidation reaction. Vermiculite is hydrated magnesium aluminum silicate that expands when heated, and is a light, highly absorbent, chemically inert, odorless, and fire-resistant material that helps diffuse the iron powder to disperse heat generated by the oxidation reaction. The nasal insert is hermetically sealed prior to use in a separate pouch to prevent oxygen from reaching the iron powder until it is time for use.

When it is time for use, the hermetically sealed pouch is opened and the nasal insert 6 is removed from the pouch. The exothermic reaction begins when the nasal insert 6 is removed from the hermetically-sealed pouch, and air begins to flow through the microporous pouch or material in the nasal insert 6. The rate of the chemical reaction and the amount of heat generated are dictated by several factors including but not limited to the porosity of the pouch, the breathing rate of the user, and the size of the lumen through the insert after it is inserted in the patient; more air and moisture exchanged through the microporous pouch or material in the nasal insert 6 causes higher temperatures. In this way, by controlling the porosity of the microporous pouch, the temperature of the exothermic reaction is controlled to be within the therapeutic range of 46° C. to 52° C. The nasal insert 6 is placed in the patient's nose. That exothermic reaction continues in the nasal cavity; as the patient breathes through the lumen in the nasal insert 6, air and water vapor in the patient's breath continues to promote the oxidation of the iron powder in the nasal insert 6. The nasal insert 6 is maintained in the nasal cavity of the patient for a sufficient time to cause cessation of bleeding.

Another example of an exothermic chemical reaction that may be used to elevate the cover 19 or other outer material of the nasal insert 6 to the therapeutic temperature range is a reaction between magnesium, iron, and water or water vapor. It is estimated that approximately one gram of magnesium and iron in combination is sufficient to generate heat within the therapeutic range of 46° C. to 52° C. for a duration sufficient for effective treatment of the patient. As with the oxidation of iron described above, vermiculite, activated charcoal, water, and salt may be combined with the magnesium and iron in a microporous pouch to control the rate of reaction. The nasal insert 6 is configured substantially as described above wherein the central lumen is configured to vent the hydrogen gas that is a byproduct of the reaction. Also substantially as described above, the exothermic reaction begins when the nasal insert 6 comes in contact with water or water vapor such as from the breath of the patient. As the patient breathes through the lumen in the nasal insert 6, air and water vapor in the patient's breath continues to promote the reaction between magnesium and iron in the nasal insert 6. Alternatively, the insert may be soaked in water prior to insertion into the patient's nose, or the insert may be inserted into the patients nose and water may be inserted into the nose to start the chemical reaction.

A third embodiment using an exothermic chemical reaction utilizes the heat of fusion of a supercooled liquid. Sodium acetate dissolved in water is an example of a stable, supercooled liquid that “freezes” at 54° C. It readily exists as a liquid at lower temperatures and is extremely stable. The exothermic phase change may be initiated by introducing foreign particles to cause a small amount of sodium acetate to crystallize. This causes the rest of the liquid to solidify as well, and the temperature of the solidifying liquid increases to 54° C. in the process. The supercooled sodium acetate is held within a reservoir inside the nasal insert 6. The foreign particles may be introduced by snapping a piece of spring steel that has a slit, where that spring steel is stored in the reservoir. The edges of the slit rub against each other and liberate small pieces of steel into the sodium acetate solution, thereby initiating the freezing process. While 54° C. is slightly above the therapeutic range of 46° C. to 52° C., that temperature is still effective for treatment; further, as the nasal insert 6 cools as the exothermic reaction ends, the temperature of the nasal insert 6 decreases to within the therapeutic range of 46° C. to 52° C. The nasal insert 6 is placed in the patient's nose, and maintained there for a sufficient time to cause cessation of bleeding.

Operation

Referring to FIGS. 1-3, the operation of the nasal insert system 1 now will be described. The nasal insert 6 may be wetted thoroughly to saturate the cover 19 with water, saline or other suitable fluid. Where the cover 19 is fabricated from, coated at least in part by, or impregnated with lubricious material such as carboxymethyl cellulose that absorbs water when the cover 19 is soaked prior to insertion into the nose, the combination of the water and the lubricious material associated with the cover 19 provides a slippery surface to improve patient comfort during insertion of the nasal insert 6. Alternately, the lubricious material may be omitted from the cover 19, and the cover 19 is lubricated with any standard lubricant to facilitate insertion. The nasal insert 6 is inserted into the nasal cavity 20 wholly or partially, with the cord 5 extending outward from the nasal cavity 20. The plug 4 at the end of the cord 5 is inserted into the plug receptacle 3, causing the control unit 2 to turn on. Alternately, the plug 4 at the end of the cord 5 is inserted into the plug receptacle 3, and the control unit 2 is turned on via a switch or in another manner. Alternately, the nasal insert 6 is inserted at least partially into the nasal cavity 20 after the plug 4 has been inserted into the plug receptacle 3 to turn on the control unit 2, or after the control unit 2 otherwise has been turned on.

Electricity flows from the control unit 2 to the resistive wires 9, causing the nasal insert 6 to begin to heat tissue to a temperature within the therapeutically effective range of 46° C. to 52° C. Optionally, such heating pauses at a lower temperature, such as 42° C., to allow the patient to acclimatize to the heat before increasing the temperature of the tissue to be treated by the nasal insert to a level between 46° C. to 52° C. In order to heat the tissue to be treated by the nasal insert to a level between 46° C. to 52° C., the nasal insert 6 may be heated to a temperature between 48° C. to 54° C., in an exemplary embodiment. Advantageously, the controller 13 within the control unit 2 periodically receives information from the thermocouple 10 or other temperature sensor as described above. As one example, the controller 13 receives information from the thermocouple 10 or other temperature sensor every few seconds, with no more than 20 seconds between such receipts of information. As another example, more than 20 seconds may pass between at least two such receipts of information. The control unit 2 controls at least one characteristic of electrical power transmitted to the resistive wires 9 based on data received from the thermocouple 10 or other temperature sensor. As one example, the control unit 2 adjusts the amount of power transmitted to the resistive wires 9 accordingly, raising the amount of power if the temperature is low, and reducing the amount of power if the temperature is high. The control unit 2 may adjust the amount of current, the amount of voltage, or both that is transmitted to the resistive wires 9 in order to control the temperature of the nasal insert 6. Alternately, the amount of power flowing from the control unit 2 to the resistive wires 9 is constant, and temperature is adjusted by turning off the power when the temperature becomes too high, and turning on the power when the temperature becomes too low. That is, the control unit 2 may adjust the duty cycle of the electrical power transmitted to the resistive wires 9 to control the temperature of the nasal insert 6. In embodiments in which the cover 19 is associated with a hemostatic agent or a chemical that causes a chemical reaction to generate heat, or both, such hemostatic agent and/or chemical reaction occurs in parallel with the administration of heat from the nasal insert 6 resulting from heat generated by application of power to the resistive wire 9. Alternately, as described above, in embodiments in which the cover 19 is associated with a hemostatic agent or a chemical that causes a chemical reaction to generate heat, or both, application of electricity to the nasal insert 6 may be omitted. Different patients will have differently-sized nasal cavities, and the nasal insert 6 may apply a small amount of pressure to the nasal cavity after its insertion. However, the treatment modality is the application of heat, as described above. The nasal insert 6 does not expand to apply force to the nasal cavity; any force or pressure applied by the nasal insert 6 to tissue in use is incidental and does not play a part in the treatment of that tissue.

Treatment continues for a therapeutically effective time. For many patients, relief from epistaxis occurs within five minutes of commencing treatment. According to some embodiments, treatment generally is provided for up to sixty minutes, which is sufficient to substantially stop the flow of blood occurring via epistaxis and provide additional therapy time to ensure bleeding stops and the healing process commences. The nasal insert 6 is removed from the nasal cavity 6. While some clotting to the nasal insert 6 may occur, the nasal insert 6 is removed before it clots in place. According to some embodiments, two nasal inserts 6 may be used at the same time in order to treat both nasal cavities simultaneously. If so, each nasal insert 6 may be connected to a different control unit 2, or both nasal inserts 6 may be connected to and controlled by the same control unit 2. Where both nasal inserts 6 are connected to the same control unit, two plug receptacles 3 are provided, and the controller 13 controls the flow of power to and monitors the temperature of both nasal inserts 6. Alternately, if bleeding has substantially ceased after treatment of one nasal cavity, the other bilateral nasal cavity may then be treated. If bleeding continues, the patient may be treated again in that nasal cavity 20, and treatment may be performed for a longer duration.

Another embodiment provides apparatus for arresting bleeding in a body cavity comprising a delivery means and an inflatable balloon connectable to the delivery means wherein the balloon is filled with a fluid in the range of 46° C. to 52° C. The fluid may be circulated within the balloon to maintain the balloon within the therapeutic temperature range.

Preferably, the balloon has a soft pliable wall made from a non-elastomeric polymeric material. The non-elastomeric balloon can be filled with fluid at a low pressure, thereby conforming to the anatomy and applying the warmth from the balloon evenly to the target tissue.

It is well known to a skilled person in the art that all plastic polymers are elastic to some extent in the strict definition of the word, that is, they obey Hooke's Law and have the ability to return to their original shape after being deformed. However, it is the extent to which the polymers can be deformed which distinguishes non-elastomeric polymeric materials from elastomeric polymeric materials. The term “elastomeric polymeric material” means a polymeric material that at room temperature can be stretched to at least twice its original length, and upon immediate release of the stress quickly returns to approximately its original length. Examples of elastomeric polymeric materials include rubber and silicon rubber. The term “non-elastomeric polymeric material” means polymeric materials which, although flexible, do not fall within the functional definition given above for elastomers. Examples of non-elastomeric polymeric material include nylon.

Preferably, the balloon has a fixed volume which ensures that the pressure in the balloon is independent of the volume of the balloon. The fixed volume non-elastomeric polymeric balloon of the device of the invention ensures that adverse effects associated with wall elasticity of known elastomeric balloons are eliminated or at least substantially mitigated, as all of or most of the pressure within the balloon is directly applied to the wall of the body cavity.

Preferably, the diameter, length and volume of the balloon is designed to be slightly greater than that of the largest cavity likely to be plugged during a particular surgical procedure. More preferably, the balloon has a diameter of between 2 mm and 20 mm, a length of between 3 mm and 20 mm and a volume of between 0.004 ml. and 5 ml. for plugging a diverticula and a diameter of between 10 mm and 75 mm, a length of between 5 mm and 100 mm and a volume of between 0.5 ml.sup.3 and 450 ml.sup.3 for plugging a bleeding nose. Balloon devices for other body cavities such as the colon, trachea or esophagus may be even larger. The appropriate dimensions of these balloon devices for a specific body cavity will be recognized by those skilled in the art, and generally consistent with the range of oversizing described above.

According to another embodiment, the fluid temperature may be increased by placing the fluid in a syringe and placing the syringe in a device such as a syringe warmer. A separate luer, shaft, and balloon assembly may also be preheated to reduce heat transfer from the heated fluid to the assembly when the heated fluid in the syringe is transferred to the balloon.

After the fluid reaches the injection temperature which may be greater than the operative temperature (46° C. to 52° C.), the warmed syringe may then be removed from the syringe warmer and connected to a separate assembly comprised of a luer connector, shaft, and balloon. The balloon is then apposed to the bleeding site, inflated with warm fluid from the syringe and left for an adequate period of time to cause cessation of bleeding. After the bleeding is stopped, the warmed fluid may be withdrawn from the balloon and removed from the operative site.

Another embodiment to maintain the balloon at the therapeutic temperature is to include a heating element within the balloon to raise the temperature of the water within the balloon to the therapeutic temperature. At least one temperature sensing device may be included in or on the balloon to provide feedback to an electronic controller to maintain the temperature of the balloon at the therapeutic temperature.

For the purposes of describing and defining the present invention it is noted that the use of relative terms such as “substantially,” “generally,” “approximately,” and the like, are utilized herein to represent an inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

Exemplary embodiments of the present invention are described above. No element, act or instruction used in this description should be construed as important, necessary, critical or essential to the invention unless explicitly described as such. Although only a few of the exemplary embodiments have been described in detail herein and those skilled in the art will readily appreciate that many modifications are possible in these exemplary embodiments without materially departing from the novel teachings and advantages of this invention. Accordingly all such modifications are intended to be included within the scope of this invention. The phrase “in one embodiment” is used repeatedly. The phrase generally does not refer to the same embodiment; however, it may. The terms “comprising,” “having” and “including” are synonymous, unless the context dictates otherwise. The following illustrations of various embodiments use particular terms by way of example to describe the various embodiments, but this should be construed to encompass and provide for terms such as “method” and “routine” and the like.

Various aspects of the illustrative embodiments will be described using terms commonly employed by those skilled in the art to convey the substance of their work to others skilled in the art. However, it will be apparent to those skilled in the art that the embodiments described herein may be practiced with only some of the described aspects. For purposes of explanation, specific numbers, materials and configurations are set forth in order to provide a thorough understanding of the illustrative embodiments. However, it will be apparent to one skilled in the art that the embodiments described herein may be practiced without the specific details. In other instances, well-known features are omitted or simplified in order not to obscure the illustrative embodiments.

The characteristics and utilities of the present invention described in this summary and the detailed description below are not all inclusive. Many additional features and advantages will be apparent to one of ordinary skill in the art given the following description. There has thus been outlined, rather broadly, the more important features of the invention in order that the detailed description thereof that follows may be better understood, and in order that the present contribution to the art may be better appreciated.

In this respect, by explaining at least one embodiment of the invention in detail, it is to be understood that the invention is not limited in its application to the details of construction and to the arrangements of the components set forth in the description. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the description be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, nor is it intended to be limiting as to the scope of the invention in any way. The characteristics and utilities of the present invention described in this summary and the detailed description below are not all inclusive. Many additional features and advantages will be apparent to one of ordinary skill in the art given the detailed description.

What is claimed is:

1. A medical apparatus for treating tissue, comprising:
a heated nasal insert,
wherein said nasal insert is configured to heat tissue to be treated to a temperature in the range of substantially 46° C. to substantially 52° C. in use.

2. The medical apparatus of claim 1, further comprising a control unit electrically connected to said nasal insert, wherein said control unit comprises at least one battery and a controller connected to said at least one battery.

3. The medical apparatus of claim 2, wherein said nasal insert further comprises:
a core;
at least one resistive wire associated with said core; and
a cover over at least part of said core and said at least one resistive wire.

4. The medical apparatus of claim 3, further comprising
a cord, wherein a first end of said cord extends from said nasal insert; and
a plug connected to a second end of said cord;
wherein said control unit further comprises a plug receptacle configured to receive said plug.

5. The medical apparatus of claim 4, wherein said control unit switches on when said plug is received into said plug receptacle, and wherein electrical power flows to said nasal insert when said control unit switches on.

6. The medical apparatus of claim 4, further comprising
a temperature sensor associated with said nasal insert, and
a sensor wire in said cord, connecting said temperature sensor to said control unit;
wherein said controller controls at least one characteristic of electric power that flows to said nasal insert such that said nasal insert is configured to heat tissue to be treated to a temperature in the range of substantially 46° C. to substantially 52° C. in use.

7. The medical apparatus of claim 3, wherein said cover comprises a hemostatic agent.

8. The medical apparatus of claim 3, wherein said cover comprises carboxymethyl cellulose.

9. The medical apparatus of claim 1, wherein at least a portion of said nasal insert undergoes an exothermic reaction upon exposure to at least one of the atmosphere, liquid water, or water vapor.

10. The medical apparatus of claim 9, wherein at least a portion of said nasal insert comprises iron powder.

11. A method for inducing hemostasis in tissue of a patient, comprising:
possessing a nasal insert;
apposing said nasal insert to tissue of a patent;
heating said nasal insert to a temperature that causes tissue of a patient to be heated to a temperature in the range of substantially 46° C. to substantially 52° C.; and
maintaining the tissue of a patent treaded by said nasal insert within a temperature range of substantially 46° C. to substantially 52° C.

12. The method of claim 11, wherein said apposing comprises inserting said nasal insert at least partially into a nasal cavity of a patient.

13. The method of claim 11, wherein said nasal insert further comprises a resistive wire and a temperature sensor, further comprising a control unit electrically connected to said nasal insert; and further comprising receiving data at said control unit from said temperature sensor;
wherein said maintaining is performed by controlling at least one characteristic of said electrical power based on said data received from said temperature sensor.

14. The method of claim 13, wherein said controlling at least one characteristic of said electrical power comprises controlling the current of said electrical power.

15. The method of claim 13, said controlling at least one characteristic of said electrical power comprises controlling the duty cycle of said electrical power.

16. The method of claim 13, wherein said temperature sensor is a thermocouple; further comprising periodically stopping the flow of said electrical power to said insert and measuring the voltage across said thermocouple wherein said electrical power is stopped, wherein said receiving data is responsive to said stopping and measuring.

17. The method of claim 11, wherein said nasal insert includes a surface that comprises a substance that facilitates hemostasis; further comprising, after said apposing, facilitating hemostasis as a result of contact between said substance and tissue of the patient.

18. The method of claim 11, further comprising lubricating said insert before said apposing.

19. The method of claim 18, wherein said nasal insert includes a surface that comprises a material that becomes lubricious upon exposure to water, and wherein said lubricating comprises applying water to said material.

20. The method of claim 11, wherein said nasal insert includes a surface that comprises a material that undergoes an exothermic reaction upon exposure to blood, and wherein said maintaining comprises continuing exposure of said nasal insert to blood.
